(19) United States
(12) Patent Application Publication
(10) Pub. No.: US 2021/0401194 A1
(43) Pub. Date: Dec. 30, 2021

(54) INTERACTIVE THERAPEUTIC BLANKET SYSTEM AND METHOD OF USE

(71) Applicant: Clear View Counseling and Consulting, LLC, Centennial, CO (US)

(72) Inventor: Kimberly S. Johancen, Centennial, CO (US)

(73) Assignee: Clear View Counseling and Consulting, LLC, Centennial, CO (US)

(21) Appl. No.: 17/336,973

(22) Filed: Jun. 2, 2021

Related U.S. Application Data
(60) Provisional application No. 63/044,897, filed on Jun. 26, 2020.

(51) Int. Cl. A47G 9/02; A61F 7/00; A61M 21/02

(52) U.S. Cl. CPC A47G 9/0215; A47G 9/0223; A61M 2021/0022; A61M 21/02; A61F 2007/0093; A61F 7/0097

(57) ABSTRACT

An interactive therapeutic blanket system is disclosed. The interactive therapeutic blanket system provides a set of selectable features that serve to address mental health therapeutic user needs. In one embodiment, a user selects a selectable blanket configuration using a system interface and a blanket system controller controls a set of active blanket devices to the selectable blanket configuration. In one feature, the set of active blanket devices include a set of vibration devices operating according to a temporally-referenced vibration regimen. In another feature, the set of active blanket devices include a set of pressure devices to impart discrete pressure to a user at discrete body locations.

### CROSS-REFERENCE TO RELATED APPLICATION

This application is a nonprovisional patent application of and claims the benefit of U.S. Provisional Patent Application No. 63/044,897 titled “Interactive Therapeutic Blanket System and Method of Use” and filed Jun. 26, 2020, the disclosure of which is hereby incorporated herein by reference in entirety.

### FIELD

The disclosure relates generally to therapeutic systems and methods of use, in particular to an interactive therapeutic blanket system and method of use.

### BACKGROUND

Therapeutic systems vary widely in type, character, and use. Examples include phototherapy using supplemental light to treat seasonal affective disorder and weighted blankets to treat anxiety. The term “therapeutic” means of or relating to the treatment of disorders through devices, systems, and/or methods.

Conventional therapeutic systems are not typically adaptable to patient-specific conditions nor to therapist-recommended settings. For example, a weighted blanket is manufactured of one uniform density throughout, providing one set pressure to the user. The pressure may not be adjusted based on user location (e.g., more pressure on torso than leg) or temporally tuned (e.g., more pressure at start of a nap or therapy session, less pressure over time). Those therapeutic systems that are adjustable to user or therapist settings are commonly limited in range or type of settings, use analog inputs or those not engaged with modern portable devices (e.g., smartphones or tablets) and associated graphical user interfaces, and not networked or otherwise coupled such that historical usage may be tracked or analyzed.

Conventional therapeutic systems are typically isolated systems that provide a single therapeutic means or feature. For example, a weighted blanket solely produces a uniform pressure on the user, the pressure commonly reducing anxiety of the user. Other physical treatments for a user suffering from anxiety, such as vibration systems attached to a user, are employed as separate, isolated systems. Furthermore, conventional therapeutic systems allow little if any tuning or adjustment to the particular needs or character of the user, as set by the user and/or a therapist. Instead, a one-size-fits-all, static therapeutic system is provided, thereby limiting the use of the therapeutic system and ultimately limiting the therapeutic impact of the system.

What is needed is a therapeutic system that integrates multiple functions or features to address the therapeutic requirements of the user and/or therapist, such a system interacting with the user and/or therapist for increased comfort and therapeutic effect, and tunable for a particular user's needs or preferences.

The disclosure solves these needs. In one embodiment, an interactive therapeutic blanket is disclosed, the interactive therapeutic blanket providing a set of selectable features that serve to address mental health therapeutic user needs.

### SUMMARY

An interactive therapeutic blanket system and method of use is disclosed. The interactive therapeutic blanket device or system provides a set of selectable features that serve to address mental health therapeutic user needs.

A user may select a selectable blanket configuration using a system interface and a blanket system controller controls a set of active blanket devices to the selectable blanket configuration. The set of active blanket devices may include, for example, a set of vibration devices operating according to a temporally-referenced vibration regimen. The set of active blanket devices may include, for example, a set of pressure devices to impart discrete pressure to a user at discrete body locations.

The interactive therapeutic may be considered a smart blanket device or smart blanket system in that the blanket incorporates one or more functions of sensing, actuation or operation, and control to describe and analyze the state or condition of the blanket (and its associated user) to make decisions or take actions based on that state or condition. The decisions or actions may be based on any control means known to those in the art, to include predictive control techniques such as closed loop control, adaptive control, stochastic control, and may employ technologies associated with neural networks, machine learning, and/or artificial intelligence. In one embodiment, an interactive therapeutic blanket device is disclosed, the device comprising: a blanket body configured to engage a user, the blanket body comprising a set of controllable blanket devices; at least one user sensor configured to provide a set of user measurements; a user system interface configured to receive a set of user inputs defining a user-selected blanket device configuration; a system controller configured to receive the set of blanket sensor measurements and the set of user measurements; and a user database operating to store a blanket device operating history, the blanket device operating history comprising a time-based record of the set of user measurements and the user-selected blanket device configuration; wherein: the system controller controls the set of controllable blanket devices to the user-selected blanket device configuration.

In one aspect, the device further comprises a user catalog of user-selected blanket device configurations, the user catalog selectable by a user using the user system interface. In another aspect, the device further comprises a therapist system interface configured to receive a set of therapist inputs defining a therapist-selected blanket device configuration. In another aspect, the system controller is configured to reconcile one or more inconsistencies between the user-selected blanket device configuration and the therapist-selected blanket device configuration to create a reconciled blanket device configuration, the controller configured to control the set of controllable blanket devices to the reconciled blanket device configuration. In another aspect, the blanket body further comprises a set of discrete thermal devices configured to transfer heat to the user at one or more discrete user body locations, the set of discrete thermal devices controlled by the system control according to a discrete thermal device control regimen, the user-selected blanket device configuration further comprising the discrete thermal device control regimen and the blanket device operating history further comprising the discrete thermal device control regimen. In another aspect, the blanket body further comprises a therapist catalog of therapist-selected blanket device configurations. In another aspect, the set of controllable blanket devices comprise a set of vibration devices. In another aspect, the set of vibration devices are a set of detachable vibration devices disposed within the blanket body or on a surface of the blanket body. In another aspect, the at least one user sensor is at least one of a user body temperature sensor, a user blood pressure sensor, and a user heart rate sensor. In another aspect, the user-selected device configuration comprises a temporally-referenced vibration regimen defining operation of the set of vibration devices. In another aspect, the set of controllable blanket devices further comprises a set of pressure devices configured to impart a first pressure at a first user body location and impart a second pressure at a second user body location, the first pressure different that the second pressure. In another aspect, the blanket body is sized to engage a child user below aged 18.

In another embodiment, a therapeutic blanket system is disclosed, the system comprising: a blanket body configured to engage a user, the blanket body comprising a set of controllable blanket devices, the set of controllable blanket devices comprising a set of detachable vibration devices coupled to the blanket body; a user system interface configured to receive a set of user inputs defining a user-selected device configuration, the user-selected device configuration including a temporally-referenced vibration regimen defining operation of the set of vibration devices, the user-selected device configuration selected from a catalog of user-selected device configurations; a system controller controlling the set of vibration devices according to the user-selected device configuration; and a user database operating to store a device operating history, the device operating history comprising a time-based record of the user-selected device configuration and an operation of the set of vibration devices.

In one aspect, the system further comprises one or more user sensors configured to provide a set of user measurements, the one or more sensors including at least one of a user body temperature, a user blood pressure, and a user heart rate, the device operating history further comprising the set of user measurements. In another aspect, the set of controllable blanket devices further comprises a set of pressure devices configured to impart a first pressure at a first user body location and impart a second pressure at a second user body location, the first pressure different that the second pressure.

In yet another embodiment, a method of using a therapeutic blanket device is disclosed, the method comprising: providing a therapeutic blanket device comprising: a blanket body configured to engage a user, the blanket body comprising a set of controllable blanket devices, the set of controllable blanket devices comprising a set of detachable vibration devices coupled to the blanket body; a user system interface; a system controller; and a user database; selecting a selectable blanket device configuration using the system interface, the selectable blanket device configuration including a temporally-referenced vibration regimen defining operation of the set of vibration devices; controlling, by way of the system controller, the set of detachable vibration devices according to the selectable blanket device configuration; and storing, on the user database, a blanket device operating history, the blanket device operating history comprising a time-based record of the selectable blanket configuration and an operation of the set of detachable vibration devices.

In one aspect, the blanket body further comprising one or more user sensors configured to provide a set of user measurements, the one or more sensors including at least one of a user body temperature, a user blood pressure, and a user heart rate, the blanket device operating history further comprising the set of user measurements. In another aspect, the method further comprises a therapist system interface configured to receive a set of therapist inputs defining a therapist-selected blanket device configuration, wherein the system controller is configured to reconcile one or more inconsistencies between the user-selected blanket device configuration and the therapist-selected blanket device configuration to create a reconciled blanket device configuration, the controller configured to control the set of controllable blanket devices to the reconciled blanket device configuration. In another aspect, the set of controllable blanket devices further comprises a set of pressure devices configured to impart a first pressure at a first user body location and impart a second pressure at a second user body location, the first pressure different that the second pressure. In another aspect, the user system interface is a portable electronic device.

By way of providing additional background, context, and to further satisfy the written description requirements of 35 U.S.C. § 112, the following references are incorporated by reference in entirety: U.S. Pat. No. 6,142,963 to Black; U.S. Pat. No. 6,170,602 to Mann; U.S. Pat. No. 6,675,743 to Jeffrey; U.S. Pat. No. 7,614,099 to Goetz; and U.S. Pat. No. 4,005,703 to Rosen; WIPO Pat. Appl. Nos. 2014/166557 to Christiansen; and 2017/194456 to Ramer.

The word “app” or “application” means a software program that runs as or is hosted by a computer, typically on a portable computer, and includes a software program that accesses web-based tools, APIs and/or data.

The term “state” means a group of variables or characteristics that defines the condition of an entity, such as pressure and temperature may define the condition or state of a substance as a gas or a liquid.

The phrase “user interface” or “UI”, the phrase “system interface,” and the phrase “graphical user interface” or “GUI”, means a computer-based display that allows interaction with a user, with aid of text, a keyboard, images, and/or graphics.

The phrase “cloud computing” or the word “cloud” refers to computing services performed by shared pools of computer resources, often over the Internet.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The term “automatic” and variations thereof, as used herein, refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material”.

The terms “determine”, “calculate” and “compute,” and variations thereof, as used herein, are used interchangeably and include any type of methodology, process, mathematical operation or technique.

The term “means” as used herein shall be given its broadest possible interpretation in accordance with 35 U.S.C., Section 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

The term “computer-readable medium” as used herein refers to any storage and/or transmission medium that participate in providing instructions to a processor for execution. Such a computer-readable medium is commonly tangible, non-transitory, and non-transient and can take many forms, including but not limited to, non-volatile media, volatile media, and transmission media and includes without limitation random access memory (“RAM”), read only memory (“ROM”), and the like. Non-volatile media includes, for example, NVRAM, or magnetic or optical disks. Volatile media includes dynamic memory, such as main memory. Common forms of computer-readable media include, for example, a floppy disk (including without limitation a Bernoulli cartridge, ZIP drive, and JAZ drive), a flexible disk, hard disk, magnetic tape or cassettes, or any other magnetic medium, magneto-optical medium, a digital video disk (such as CD-ROM), any other optical medium, punch cards, paper tape, any other physical medium with patterns of holes, a RAM, a PROM, and EPROM, a FLASH-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored. Computer-readable storage medium commonly excludes transient storage media, particularly electrical, magnetic, electromagnetic, optical, magneto-optical signals.

Moreover, the disclosed methods may be readily implemented in software and/or firmware that can be stored on a storage medium to improve the performance of: a programmed general-purpose computer with the cooperation of a controller and memory, a special purpose computer, a microprocessor, or the like. In these instances, the systems and methods can be implemented as program embedded on personal computer such as an applet, JAVA® or CGI script, as a resource residing on a server or computer workstation, as a routine embedded in a dedicated communication system or system component, or the like. The system can also be implemented by physically incorporating the system and/or method into a software and/or hardware system, such as the hardware and software systems of a communications transceiver.

Various embodiments may also or alternatively be implemented fully or partially in software and/or firmware. This software and/or firmware may take the form of instructions contained in or on a non-transitory computer-readable storage medium. Those instructions may then be read and executed by one or more processors to enable performance of the operations described herein. The instructions may be in any suitable form, such as but not limited to source code, compiled code, interpreted code, executable code, static code, dynamic code, and the like. Such a computer-readable medium may include any tangible non-transitory medium for storing information in a form readable by one or more computers, such as but not limited to read only memory (ROM); random access memory (RAM); magnetic disk storage media; optical storage media; a flash memory, etc.

The preceding is a simplified summary of the disclosure to provide an understanding of some aspects of the disclosure. This summary is neither an extensive nor exhaustive overview of the disclosure and its various aspects, embodiments, and/or configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and/or configurations of the disclosure are possible utilizing, alone or in combination, one or more of the features set forth above or described in detail below. Also, while the disclosure is presented in terms of exemplary embodiments, it should be appreciated that individual aspects of the disclosure can be separately claimed.

### BRIEF DESCRIPTION OF THE DRAWINGS

The disclosure will be readily understood by the following detailed description in conjunction with the accompanying drawings, wherein like reference numerals designate like elements. The elements of the drawings are not necessarily to scale relative to each other. Identical reference numerals have been used, where possible, to designate identical features that are common to the figures.

FIG. 1 is a schematic representation of one embodiment of interactive therapeutic blanket system;

FIG. 2A is a schematic representation of one embodiment of the blanket component of the interactive therapeutic blanket system of FIG. 1, detailing the active blanket devices;

FIG. 2B is a schematic representation of one embodiment of the blanket component of the interactive therapeutic blanket system of FIG. 1, detailing the blanket sensor devices;

FIG. 2C is a schematic representation of one embodiment of the blanket component of the interactive therapeutic blanket system of FIG. 1, detailing the user sensors coupled with active blanket devices; and

FIG. 3 is a flow chart of one method of use of the interactive therapeutic blanket system of FIG. 1.

### DETAILED DESCRIPTION

Reference will now be made in detail to representative embodiments. The following descriptions are not intended to limit the embodiments to one preferred embodiment. To the contrary, it is intended to cover alternatives, modifications, and equivalents as can be included within the spirit and scope of the described embodiments as defined, for example, by the appended claims.

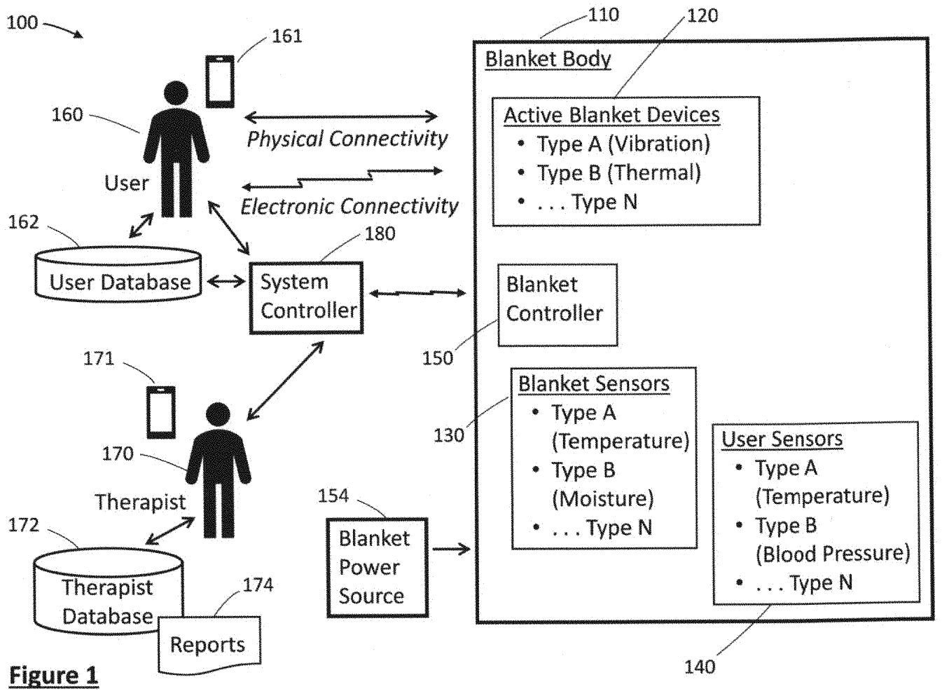

The disclosed devices, systems, and methods of use will be described with reference to FIGS. 1-3. Generally, systems and methods to provide an interactive therapeutic blanket system are provided.

Although the disclosed devices, systems, and methods of use will be described relative to an interactive therapeutic blanket system, the devices, systems, and methods of use have other applications. For example, the interactive therapeutic blanket system may be used to provide interactive therapeutic features and/or functions by way of clothing worn by a user, such as a jacket. Other applications or uses are possible.

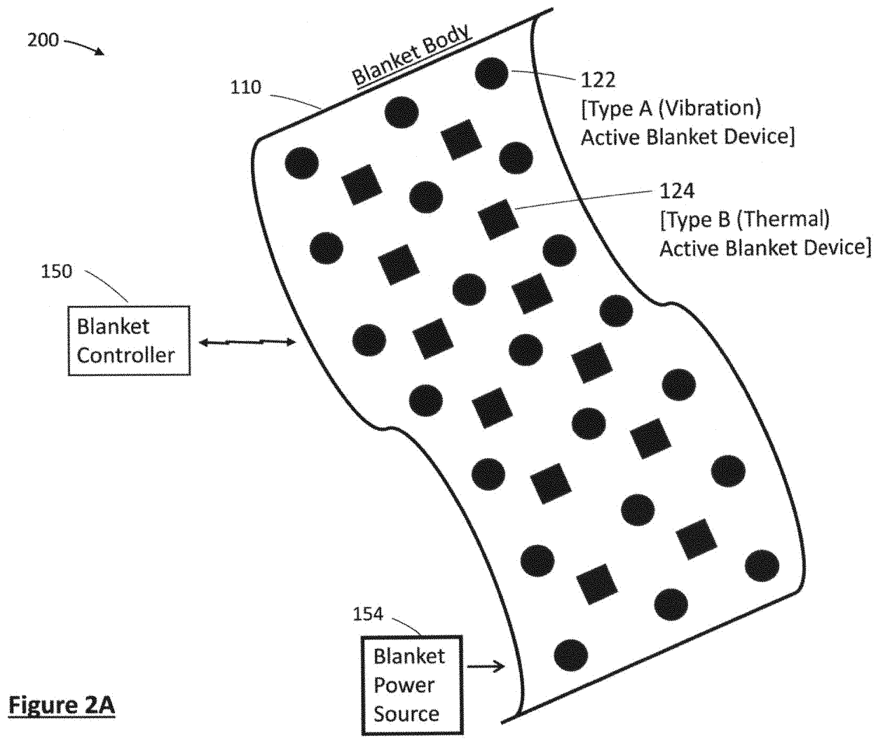

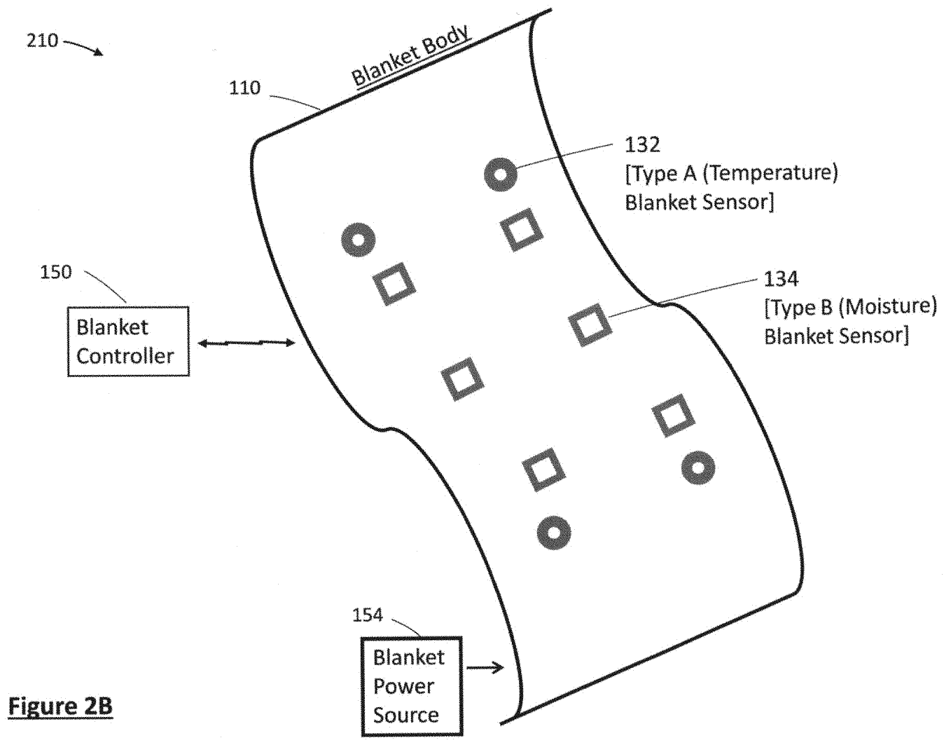

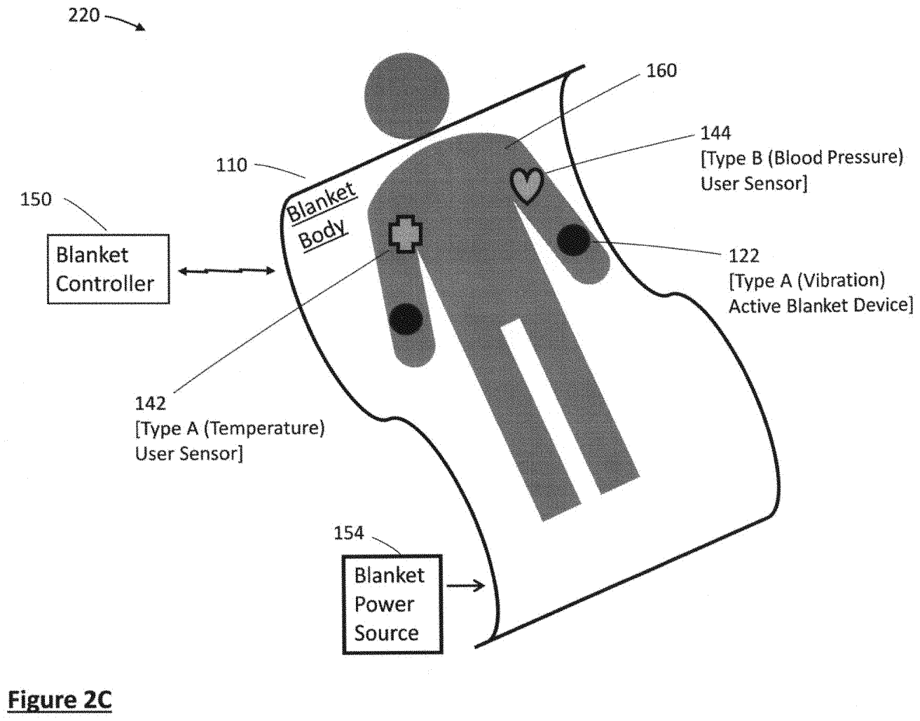

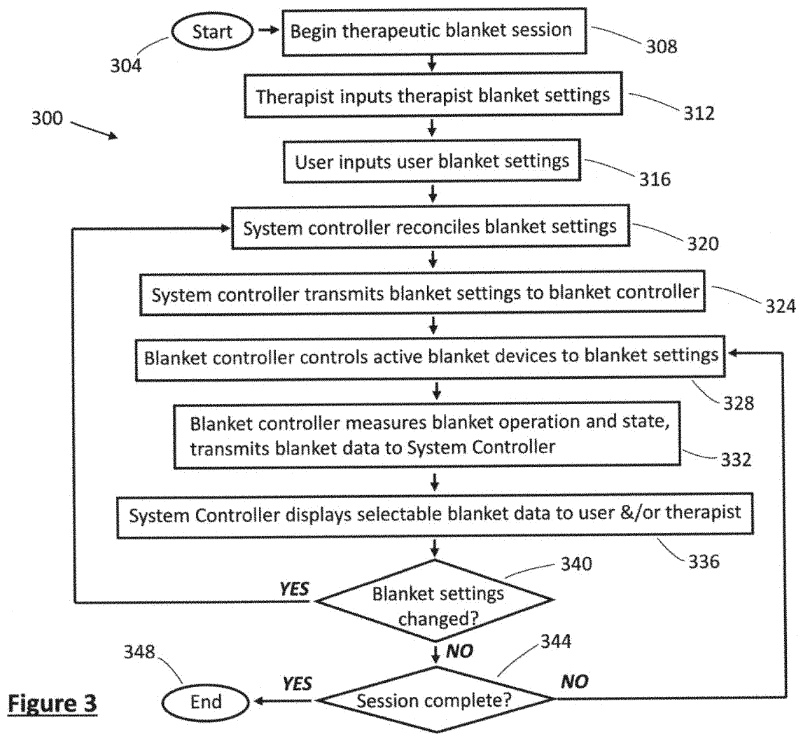

With attention to FIG. 1, an interactive therapeutic blanket system 100 is depicted. FIGS. 2A-C provide further details of the blanket component of the interactive therapeutic blanket system aka interactive therapeutic blanket device of FIG. 1. FIG. 3 provides a method of using the interactive therapeutic blanket system 100 of FIG. 1. Other methods of use are possible. The interactive therapeutic blanket system 100 may also be referred to as “system,” “blanket system,” “device,” “blanket device,” “therapeutic system,” “therapeutic device,” “therapeutic blanket device” and/or “therapeutic blanket system.” The method of using the interactive therapeutic blanket system 100 may also be referred to as “method.”

Generally, a therapeutic blanket system is provided that may be configured by a user and/or a therapist to provide therapeutic attention to a user. The therapeutic attention is provided by a set of active blanket devices, such as vibration or thermal (heat) elements, positioned on or within the blanket or positioned on or very near the user. The active blanket devices may be referred to as coupled to the blanket and/or blanket body, meaning the active blanket devices are engaged with the blanket and/or blanket body. The blanket may also be fitted with blanket sensors to measure blanket state, e.g., blanket temperature or blanket moisture. The blanket sensors may be considered to be coupled to the blanket and/or blanket body. The user may also be engaged with user sensors that provide measurements of user parameters, such as user blood pressure, heart rate, or user temperature. The user sensors may be considered to be coupled to the user's body, such as engaged with a user's wrist, neck, torso, arm, leg, etc. The various sensors or blanket devices are controlled by a system controller and/or a blanket controller, which control the blanket system to the configuration selected by the user and/or the therapist. The configuration may be selected and/or monitored by way of a user device, such as a smartphone or tablet computer. Historical data may be recorded to a system database to create a blanket device operating history, and user preferences may be stored or accessed to a user database. Therapist created therapeutic regimens may be stored in a therapist database, which enables customizable (to a particular user per therapist requirements) regimens to be activated. The therapeutic regimens may also utilize integrated therapeutic techniques (e.g., vibration, acoustic, thermal, etc.) to be employed. In one embodiment, the user database and the therapist database are combined into one database.

With attention to FIGS. 1 and 2A-C, the therapeutic blanket system 100 comprises a blanket body 110, a system controller 180, at least one system interface 161, 171, and a user 160. The blanket body 110 may comprise a blanket controller 150, a blanket power source 154, and a set of active blanket devices 120. A user 160, such as a user with mental health therapeutic needs, engages the therapeutic blanket system 100 by way of user system interface 161. In some embodiments, the blanket body 110 further includes one or more of a set of blanket sensors 130 and a set of user sensors 140. In some embodiments, the therapeutic blanket system 100 also includes a therapist 170, the therapist engaging the therapeutic blanket system 100 by way of therapist user interface 172. The user 162 may engage a user database 162. The therapist may engage a therapist database 172, the therapist database may include or interact with a reporting system 174.

It is important to note that, as provided above, embodiments and/or configurations of the therapeutic blanket system 100 are possible utilizing, alone or in combination, one or more of the features set forth above or described in detail below. Stated another way, the various features or elements of the therapeutic blanket system 100 may be combined, added to, or deleted from to create new embodiments. For example, in one embodiment, the blanket body 110 does not include any blanket sensors 130 and does not include any user sensors 140 but does include active blanket devices 120. More specifically, in one embodiment the blanket includes thermal devices (to heat the blanket) and vibration devices and is a weighted blanket, the blanket configuration established or set by a user through a user interface (to set, e.g., vibration settings or modes with time) and controlled automatically through a control system. In one embodiment, the therapist 170 is more broadly a second user or otherwise interested party.

FIGS. 2A-C provide schematic representations of embodiments of the blanket 200, 210, 220 of the interactive therapeutic blanket system 100 of FIG. 1, each representation comprising a blanket controller 150 and blanket power source 154. FIG. 2A details a blanket 200 with active blanket devices 122, 124. FIG. 2B details a blanket 210 with blanket sensors 132, 134. FIG. 2C details a blanket 220 with both active blanket devices 122 and user sensors 142, 144.

Note that the configurations of the blanket body 110 component of the interactive therapeutic blanket system 100, as depicted in FIGS. 2A-C, are example configurations and are not limiting; other configurations are possible. Other configurations that involve different collections of sensors and/or devices, in different physical locations relative to the blanket body 110, are possible. Also, the various sensors and/or devices may be positioned in any of several ways, to include within a thickness of the blanket body 110, partially within a thickness of the blanket body 110, adjacent the blanket body 110, on a surface of the blanket body 110, or, in some cases (see FIG. 2C for example), positioned on or adjacent a user 160. One or more of the active blanket devices 122 and user sensors 142, 144 may be detachable such that the user may re-position or re-orient the active blanket devices 122 and/or user sensors 142, 144 to different locations relative to the blanket body and/or user.

Each of the user 160 and the therapist 170 engage with the therapeutic blanket system 100 by way of a system interface 161, 171 respectively. The system interface 161, 171 may be any device that allows input of therapeutic blanket system 100 parameters, such as selectable blanket configuration. The system interface 161, 171 may be an app assessable by a portable device such as a laptop, smartphone, or tablet. The selectable blanket configuration may, for example, include settings for the various sensors and devices of the blanket body 110, either in a static mode (a set value that remains unless changed) or a dynamic mode (a value that change with time or condition). The user-selectable blanket device configuration may be selectable with use of a user database 162 accessed by the user 160 and/or therapist 170, and/or a therapist database 172 accessed by the therapist 170. The databases 162, 172 may comprise preset programs or regimens of therapeutic blanket system 100 parameters or settings of selectable blanket configurations, such as a sequence over a set time involving a set of active blanket devices 120. In one embodiment, the set of active blanket devices may be configured or set to operate independently of one another, e.g., a set of vibration devices may be configured or set to operate independently of a set of thermal devices.

The blanket power source 154 may be a battery, to include a rechargeable battery, or any power source known to those skilled in the art, to include direct plug in to an electrical power source such as a residential electricity wall socket.

As an example, selectable blanket configurations that defines a therapeutic regimen, the therapeutic blanket system 100 may be configured to operate with a selectable set of vibration active devices 122 operating at a set frequency and amplitude (strength) for 60 min, then a different set of frequency and amplitude for the next 60 min, with the blanket temperature set and controlled through a selectable set of thermal active devices 124. More specifically, the therapeutic blanket system may be configured or set in a first configuration (e.g., a relatively slow vibration) that reduces stress or to amplify resourcing (so as to assist a user in getting through painful feelings/activation); such a first configuration may subsequently be associated by the user with relaxation. Such mappings of blanket configuration with user state or condition may be stored in one or more databases of the system and may create a historical record of user interactions with the therapeutic blanket system.

A particular selectable blanket configuration, as input by one or both of the user 160 by way of user system interface 161 and by therapist 170 by way of therapist system interface 171 (to provide a therapist-selected blanket device configuration, is transmitted to system controller 180. The system controller 180 arbitrates or reconciles any inconsistencies between parameters of a selectable blanket configuration input by each of the therapist 170 and user 160, according to a selectable rule set or through interaction or negotiation between the therapist and the user. The resulting integrated or merged blanket device configuration may be deemed a reconciled blanket device configuration. The rule set may be as simple as any conflict is resolved in favor or the therapist settings or instead the user settings. Other rules sets are possible, e.g., as a function of a particular parameter. For example, the user may be able to set length of session or temperature of blanket, but all other parameters are set by the therapist.

The system controller 180 generates control signals for implementation by components of the blanket body 110, such as the various sensors and devices (e.g., elements 120, 130, 140) of the blanket body 110. The control signals of the system controller 180 may be provided directly to the various sensors and devices or as shown in FIG. 1 or may be provided to blanket controller 150. In one embodiment, the blanket controller 150 receives the selectable blanket configuration from one or both of the user 160 and therapist 170 and performs the functions of the system controller 180, thereby removing the system controller 180 from the therapeutic blanket system 100.

The control signals required to control or operate the blanket body 110 at the desired selectable blanket configuration are transmitted to the various sensors and devices of the blanket body 110. For example, in the blanket configuration of FIG. 2A, the blanket controller 150 would send control signals to the set of type A (vibration) active blanket devices 122 to operate in the selected manner (e.g., frequency and amplitude/strength of vibration for each of several vibration devices) and to the set of type B (thermal) active blanket devices 124 in the selected (e.g., at a selected temperature). It should be noted that each of a given type of active blanket device may operate at a different configuration or a subset of active blanket devices may operate in a coordinated configuration. For example, the set of vibration elements 122 forming the outer perimeter of the blanket 122 may operate in one common manner, while the remaining vibration elements 122 operate in a different manner.

The collection of sensors and devices of the blanket body 110 may operate in a coordinated fashion by way of the system controller 180 and/or blanket controller 150 (the “controllers”) to achieve or toward achieving the selectable blanket configuration. Stated another way, the system controller 180 and/or blanket controller 150 may control the set of sensors and devices of the blanket such that the therapeutic blanket system 100 operates in the selectable blanket configuration. With attention to FIG. 2B, a set of type A (temperature) blanket sensors 132 and a set of type B (moisture) blanket sensors 134 are coupled to the blanket body 110. The blanket controller 150 operates to monitor these sensors and receive measurements of, respectively, temperature of the blanket at or near the sensor location and moisture at or near the sensor location. Note that a moisture sensor may valuable, for example, to indicate that a user may be sweating perhaps due to a fever or anxious condition. The blanket controller 150 employs feedback control, as is well known in the art, to adjust control signals based on sensor measurements. For example, the temperature sensor 132 measurements enable the blanket controller 150 to adjust control signals to the thermal active blanket devices 122 to control to a blanket temperature setting as provided in the selectable blanket configuration.

Another example embodiment of a blanket body 110 configuration is provided on FIG. 2B, wherein two types of user sensors (142, 144), coupled or in contact with a user 160, are employed along with a set of two vibration active blanket devices 122. The temperature user sensor 142 is depicted as attached or disposed on or near the armpit of user 142 so as to obtain body temperature of the user 160, and the blood pressure user sensor 144 is depicted as attached or disposed on or near an upper arm of the user 160, so as to obtain a blood pressure measurement of the user 160. Each of the two vibration active blanket devices 122 are depicted as attached or disposed on the wrists of the user 160, so as to impart vibration to the user 160. The set of devices and sensors of FIG. 2C would enable a therapeutic regimen to be provided to a user 160 that conforms to a desired selectable blanket configuration, the therapeutic regimen involving providing vibration patterns to the wrists of a user 160 while monitoring the user's blood pressure and body temperature.

A particular selectable blanket configuration may be associated with, or developed from, a particular therapeutic regimen, as stored in one or more of the user database 162 and therapist database 172. Additively or alternatively, a particular selectable blanket configuration may be associated with, or developed from, one or more reports 174 of user 160 historical therapeutic sessions. The therapeutic regimens may be related to or derived from any of a number of therapeutic regimens known to those skilled in the art to include, for example, bilateral stimulation (BLS), eye movement desensitization and reprocessing (EMDR), etc.

The blanket body 110 may comprise a variety of sensors and devices in a number of configurations. A particular configuration would be defined by number, type, and position of a set of sensors and devices. Three representative configurations of sensors and devices are provided as FIGS. 2A-C.

The active blanket devices 122 may be of several types, to include vibration devices which emits vibration, thermal devices that emit heat, cooling devices that provide a cooling effect, acoustic devices that emit a sound such as white noise, pressure devices that provide a pressure to a user, and other devices known to those skilled in the art, to include those active devices used to effect or implement a particular therapeutic regimen. For example, in one configuration, a set of vibration devices are employed to provide bilateral stimulation (BLS) to a user, such as that provided by the Touchpoint™ product. (Note that BLS means stimuli, e.g., visual, auditory or tactile, which occur in a rhythmic left-right pattern, such as moving light alternating from left to right and back again or listening to tones that alternate between the left and right sides of the head).

In another example, one or more acoustic or noise devices are provided which provide an acoustically based therapeutic sensation to the user and may interact with devices which produce a wave sensation to the user that coincides with acoustic waves or sound waves.

Note that a set of pressure devices, in one embodiment, could operate to provide different levels of pressure at different blanket locations by having a linked system of fluid between the pressure devices, such that greater fluid in a pressure device results in relatively more pressure. Such pressure devices could also be configured (and controlled by the system controller 180 and/or blanket controller 150) so as to impart greater pressure to particular locations of a user (location of the user relative to the blanket determined by, e.g., contact sensors disposed throughout the blanket). Stated another way, a set of discrete pressure devices may impart a first pressure at a first user body location and impart a second pressure at a second user body location, the first pressure different that the second pressure. Dynamic pressure devices as described (or other ways to effect pressure-by-location of the blanket) could also provide a regimen of pressure application to a user with time, not unlike the varied vibration provided to a user by way of vibration devices. Stated another way, a therapeutic regimen of blanket pressure could be applied to a user.

The blanket sensors 130 may be of several types, to include temperature sensors, moisture sensors, contact sensors, and other sensors known to those skilled in the art, to include those blanket sensors used to effect or implement a particular therapeutic regimen.

The user sensors 140 may be of several types, to include temperature sensors, blood pressure sensors, eye movement sensors such as those measuring rapid eye movement, sleep sensors such as those associated with sleep apnea, heart rate monitors, and other sensors known to those skilled in the art, to include those blanket sensors used to effect or implement a particular therapeutic regimen.

Further details of the elements of the therapeutic blanket system 100 of FIGS. 1 and 2A-C will be described in concert with a method 300 of using the therapeutic blanket system 100 as depicted in FIG. 3.

FIG. 3 is a flow chart of one method of using therapeutic blanket system 100. In one embodiment of a method of use, the therapeutic blanket system 100 follows the sequence of steps described in FIG. 3. Other methods of use are possible, to include a sequence of steps different than those of FIG. 3, a sequence with additional steps, a sequence with fewer steps, and the sequence of steps provided as FIG. 3. Also, as will be clear from the below description, elements of the therapeutic blanket system 100 of FIG. 1, and/or other aspects of a therapeutic blanket system 100 as described in this disclosure, may be incorporated.

With particular attention to FIG. 3, a flowchart of a method of use of a therapeutic blanket system is provided, the method 300 utilizing the elements described in the systems of FIG. 1 and FIGS. 2A-C.

The method 300 starts at step 304 and ends at step 348. Any of the steps, functions, and operations discussed herein can be performed continuously and automatically. In some embodiments, one or more of the steps of the method 300 may comprise computer control, use of computer processors, and/or some level of automation. The steps are notionally followed in increasing numerical sequence, although, in some embodiments, some steps may be omitted, some steps added, and the steps may follow other than increasing numerical order.

At step 308, a session using the therapeutic blanket system 100 by a user 160 begins. A therapeutic blanket system 100 as described above is provided. After completion of step 308, the method 300 continues to step 312.

At step 312, the therapist 170 inputs a desired selectable blanket configuration by way of the therapist system interface 161. The configuration may be selected based on historical user data (providing, for example, a time-based record of blanket device operation and/or configuration) as stored on one or both of the therapist database 172 and reports 174 elements. The blanket configuration may reflect or be influenced by a therapeutic regimen the therapist 170 has developed for the user 160. The blanket configuration, as described above, may vary from static values for one or more active blanket devices 120 to temporally based programs involving multiple sensors and devices. After completion of step 312, the method 300 continues to step 316.

At 316, the user 160 inputs a desired selectable blanket configuration by way of the user system interface 171. In one embodiment, the user 160 may view the blanket configuration selected by the therapist 170 and enter suggested edits to it. (In an embodiment in which the user inputs a selectable blanket configuration before the therapist 170 so enters, the therapist may view the blanket configuration selected by the user 160 and enter suggested edits to it). In one embodiment, the user 160 and the therapist 170 may both interactively and/or sequentially edit and tune the selectable blanket configuration; such a mode of operation would be particularly useful if the user 160 and therapist 170 are physically at different locations or not available simultaneously to develop a particular blanket configuration. After completion of step 316, the method 300 continues to step 320.

At step 320, the system controller 320 reconciles any conflicts in blanket settings, as caused by any of several circumstances. For example, conflicts in settings may exist in that conflicts exist in disagreements in inputs by the user 160 and the therapist, such as duration of session, use of certain devices (e.g., the user may not like use of vibration devices, yet the therapist feels vibration devices are appropriate), and the like. Other conflicts may also exist, such as inability to use a particular device due to maintenance issues (e.g., dynamic pressure devices may be inoperable). The controller creates a reconciled blanket device configuration that resolves any conflicts, e.g., conflicts created by incompatible or inconsistent user inputs and therapist inputs. After completion of step 320, the method 300 continues to step 324.

At step 324, the system controller transmits control schemes, control signals, and/or selectable blanket configuration to the blanket controller 150 to enable control or operation of the therapeutic blanket system to the selectable blanket configuration. At the completion of step 324, the method 300 continues to step 328.

At step 328, the blanket controller 150 controls the various elements of the blanket body 110, such as devices 120 and/or sensors 130, 140, to control the blanket body 110 to the selectable blanket configuration. At the completion of step 328, the method 300 continues to step 332.

At step 332, the blanket controller 150 measures blanket operation (e.g., blanket temperature), blanket state (e.g., valid operation of actives devices) and/or user state (e.g., user heart rate) and transmits such data to the system controller. (Such data are also used by the blanket controller 150 to control the blanket body 110). After completion of step 332, the method 300 continues to step 336.

At step 336, the system controller produces a display on one or both of the user system interface 161 and the therapist system interface 171 that reflects any of a number of blanket parameters, user or blanket states, and/or progress relative to the selectable blanket configuration or therapeutic session. For example, the display may present essentially raw sensor readings as to user state (e.g., heart rate, blood pressure) and active device status (e.g., vibration device 1 operating and produce 60 Hz pulses of medium amplitude/strength).

The user state data may also be correlated with blanket parameters to assess the effectiveness, or a per user basis, of blanket operation with user state. For example, a first vibration regimen may result in reduced blood pressure of a user, while a second vibration regimen may result in heightened blood pressure. As such, the collected data (presented in real-time and/or collected as historical data) allows the user or therapist to observe or gauge the effectiveness of a particular blanket configuration or therapy regimen. In other words, the user may be able to prove to themselves that they are indeed relaxing or responding to the therapeutic quality of the blanket, and if not, enable the user to re-configure the blanket accordingly. After completion of step 336, the method 300 continues to step 340.

At step 340, a query is made to determine if blanket settings have changed. For example, the user 160 or the therapist may have selected (by way of respective user system interface 161 or therapist system interface 171) to stop the therapeutic blanket session, or to decrease amplitude/strength of one or more vibration devices. If the result of the query is YES (that is, a blanket setting has changed), the method continues to step 320. If the result is NO (that is, blanket settings have not changed), the method continues to step 344.

At step 344, a query is made to determine if the therapeutic blanket session has been completed (as determined by completing the selected blanket configuration). If the result of the query is YES (that is, the therapeutic blanket session is complete), the method ends at step 348. If the result is NO (that is, the therapeutic blanket session is not complete), the method continues to step 328.

The blanket body 110 may be of any size or dimension to accommodate a user. In one embodiment, the blanket body 110 is sized to fit a typical child (defined as, e.g., a person less than 18 years old). In another embodiment, the blanket body 110 is sized to fit a typical adult. The various blanket sensors and blanket devices may also be tuned or adapted for a particular user. For example, a set of vibration sensors may provide a more limited range or reduced maximum amplitude when fitted or adapted for a child user.

Communication between elements of the therapeutic blanket system may be by any of several means, to include wireless communications. Data storage and/or access to data of the therapeutic blanket system, to include the reports 174, user database 162, and therapist database 162, may be by any of several means, to include use of the cloud.

The exemplary systems and methods of this disclosure have been described in relation to systems and methods involving an interactive therapeutic blanket system. However, to avoid unnecessarily obscuring the present disclosure, the preceding description omits a number of known structures and devices, and other application and embodiments. This omission is not to be construed as a limitation of the scopes of the claims. Specific details are set forth to provide an understanding of the present disclosure. It should however be appreciated that the present disclosure may be practiced in a variety of ways beyond the specific detail set forth herein.

A number of variations and modifications of the disclosure can be used. It would be possible to provide for some features of the disclosure without providing others.

Although the present disclosure describes components and functions implemented in the aspects, embodiments, and/or configurations with reference to particular standards and protocols, the aspects, embodiments, and/or configurations are not limited to such standards and protocols. Other similar standards and protocols not mentioned herein are in existence and are considered to be included in the present disclosure. Moreover, the standards and protocols mentioned herein, and other similar standards and protocols not mentioned herein are periodically superseded by faster or more effective equivalents having essentially the same functions. Such replacement standards and protocols having the same functions are considered equivalents included in the present disclosure.

The present disclosure, in various aspects, embodiments, and/or configurations, includes components, methods, processes, systems and/or apparatus substantially as depicted and described herein, including various aspects, embodiments, configurations embodiments, sub-combinations, and/or subsets thereof. Those of skill in the art will understand how to make and use the disclosed aspects, embodiments, and/or configurations after understanding the present disclosure. The present disclosure, in various aspects, embodiments, and/or configurations, includes providing devices and processes in the absence of items not depicted and/or described herein or in various aspects, embodiments, and/or configurations hereof, including in the absence of such items as may have been used in previous devices or processes, e.g., for improving performance, achieving ease and\or reducing cost of implementation.

The foregoing discussion has been presented for purposes of illustration and description. The foregoing is not intended to limit the disclosure to the form or forms disclosed herein. In the foregoing Detailed Description for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An interactive therapeutic blanket device comprising:
a blanket body configured to engage a user, the blanket body comprising a set of controllable blanket devices;
at least one user sensor configured to provide a set of user measurements;
a user system interface configured to receive a set of user inputs defining a user-selected blanket device configuration;
a system controller configured to receive the set of blanket sensor measurements and the set of user measurements; and
a user database operating to store a blanket device operating history, the blanket device operating history comprising a time-based record of the set of user measurements and the user-selected blanket device configuration;
wherein:
the system controller controls the set of controllable blanket devices to the user-selected blanket device configuration.

2. The interactive therapeutic blanket device of claim 1, further comprising a user catalog of user-selected blanket device configurations, the user catalog selectable by a user using the user system interface.

3. The interactive therapeutic blanket device of claim 1, further comprising a therapist system interface configured to receive a set of therapist inputs defining a therapist-selected blanket device configuration.

4. The interactive therapeutic blanket device of claim 3, wherein the system controller is configured to reconcile one or more inconsistencies between the user-selected blanket device configuration and the therapist-selected blanket device configuration to create a reconciled blanket device configuration, the controller configured to control the set of controllable blanket devices to the reconciled blanket device configuration.

5. The interactive therapeutic blanket device of claim 1, wherein the blanket body further comprises a set of discrete thermal devices configured to transfer heat to the user at one or more discrete user body locations, the set of discrete thermal devices controlled by the system control according to a discrete thermal device control regimen, the user-selected blanket device configuration further comprising the discrete thermal device control regimen and the blanket device operating history further comprising the discrete thermal device control regimen.

6. The interactive therapeutic blanket device of claim 3, further comprising a therapist catalog of therapist-selected blanket device configurations.

7. The interactive therapeutic blanket device of claim 1, wherein the set of controllable blanket devices comprise a set of vibration devices.

8. The interactive therapeutic blanket device of claim 7, wherein the set of vibration devices are a set of detachable vibration devices disposed within the blanket body or on a surface of the blanket body.

9. The interactive therapeutic blanket device of claim 7, wherein the at least one user sensor is at least one of a user body temperature sensor, a user blood pressure sensor, and a user heart rate sensor.

10. The interactive therapeutic blanket device of claim 9, wherein:
the user-selected device configuration comprises a temporally-referenced vibration regimen defining operation of the set of vibration devices.

11. The interactive therapeutic blanket device of claim 7, wherein the set of controllable blanket devices further comprises a set of pressure devices configured to impart a first pressure at a first user body location and impart a second pressure at a second user body location, the first pressure different that the second pressure.

12. The interactive therapeutic blanket device of claim 1, wherein the blanket body is sized to engage a child user below aged 18.

13. A therapeutic blanket system comprising:
a blanket body configured to engage a user, the blanket body comprising a set of controllable blanket devices, the set of controllable blanket devices comprising a set of detachable vibration devices coupled to the blanket body;
a user system interface configured to receive a set of user inputs defining a user-selected device configuration, the user-selected device configuration including a temporally-referenced vibration regimen defining operation of the set of vibration devices, the user-selected device configuration selected from a catalog of user-selected device configurations;
a system controller controlling the set of vibration devices according to the user-selected device configuration; and
a user database operating to store a device operating history, the device operating history comprising a time-based record of the user-selected device configuration and an operation of the set of vibration devices.

14. The therapeutic blanket system of claim 13, the blanket body further comprising one or more user sensors configured to provide a set of user measurements, the one or more sensors including at least one of a user body temperature, a user blood pressure, and a user heart rate, the device operating history further comprising the set of user measurements.

15. The therapeutic blanket system of claim 14, wherein the set of controllable blanket devices further comprises a set of pressure devices configured to impart a first pressure at a first user body location and impart a second pressure at a second user body location, the first pressure different that the second pressure.

16. A method of using a therapeutic blanket device, the method comprising:
providing a therapeutic blanket device comprising:
a blanket body configured to engage a user, the blanket body comprising a set of controllable blanket devices, the set of controllable blanket devices comprising a set of detachable vibration devices coupled to the blanket body;
a user system interface;
a system controller; and
a user database;
selecting a selectable blanket device configuration using the system interface, the selectable blanket device configuration including a temporally-referenced vibration regimen defining operation of the set of vibration devices;
controlling, by way of the system controller, the set of detachable vibration devices according to the selectable blanket device configuration; and
storing, on the user database, a blanket device operating history, the blanket device operating history comprising a time-based record of the selectable blanket configuration and an operation of the set of detachable vibration devices.

17. The method of claim 14, the blanket body further comprising one or more user sensors configured to provide a set of user measurements, the one or more sensors including at least one of a user body temperature, a user blood pressure, and a user heart rate, the blanket device operating history further comprising the set of user measurements.

18. The method of claim 16, further comprising a therapist system interface configured to receive a set of therapist inputs defining a therapist-selected blanket device configuration, wherein the system controller is configured to reconcile one or more inconsistencies between the user-selected blanket device configuration and the therapist-selected blanket device configuration to create a reconciled blanket device configuration, the controller configured to control the set of controllable blanket devices to the reconciled blanket device configuration.

19. The method of claim 16, wherein the set of controllable blanket devices further comprises a set of pressure devices configured to impart a first pressure at a first user body location and impart a second pressure at a second user body location, the first pressure different that the second pressure.

20. The method of claim 14, wherein the user system interface is a portable electronic device.
